(19) United States
(12) Patent Application Publication
(10) Pub. No.: US 2014/0315285 A1
(43) Pub. Date: Oct. 23, 2014

(54) LIGNOCELLULOSIC DETECTION DEVICE

(71) Applicant: National Tsing Hua University, Hsinchu (TW)

(72) Inventors: Chen-Meng KUAN, Hsinchu (TW); Hsi-Kai WANG, Hsinchu (TW); Kuan-Hung CHEN, Hsinchu (TW); Robert S. LANGER, Hsinchu (TW); Chao-Min CHENG, Hsinchu (TW)

(21) Appl. No.: 13/930,676

(22) Filed: Jun. 28, 2013

(30) Foreign Application Priority Data
Apr. 19, 2013 (TW) 102113930

(51) Int. Cl. G01N 31/22; G01N 33/84; G01N 33/66

(52) U.S. Cl. CPC G01N 31/227; G01N 33/66; G01N 33/84
USPC 435/287.7; 422/69

(57) ABSTRACT

A lignocellulosic detection device includes a lignocellulosic substrate and at least one detection reagent absorbed/coated to lignocellulosic tissue of the lignocellulosic substrate. A liquid specimen is in contact with the lignocellulosic substrate so as to be absorbed to the lignocellulosic tissue of the lignocellulosic substrate by capillary action and react with the detection reagent to achieve specimen detection. The lignocellulosic detection device of the present invention is provided with inherent advantages of the lignocellulosic substrate such as natural material, low cost, easy manipulation and capillary effect and results in a good preventive diagnosis platform. Also, users may achieve preventive disease diagnosis without spending additional time and/or money.

### BACKGROUND OF THE INVENTION

1. Field of the Invention

The present invention relates to a detection device, and particularly relates to a lignocellulosic detection device achieved by capillary action.

2. Description of the Prior Art

With the increasing trend in people's health awareness, the concept of self-testing at home has gradually prevailed. Self-test kits provide tests that people can perform tests at home at any time and may be achieved by observing the color change of the detection reagent. Therefore, disease signs can be immediately observed with simple instruments or unaided eyes instead of complex instruments. In addition, when the color changes grow larger, users may go to the hospital for further detailed examination. Therefore, self-test kits have advantages in immediate facilitation and saving money.

Regarding self-test kits used at home, test strips have been frequently used. Test strips are provided with advantages such as simple operation, easy to interpret and easy to carry. The principle of test strip detection is achieved by biochemical reactions catalyzed by the enzyme and specific substances so as to produce changes in color or other properties, and a qualitative reaction for detection the presence of a substance or a semi-quantitative reaction for determining the concentration of the substance may thus be achieved.

For example, a glucose oxidase method applied in home-use blood sugar or urine glucose test strips refers to the color changes in test strip achieved by the specificity of the catalytic reaction between glucose oxidase and glucose while other reducing substances does not work. A test strip coated with a glucose oxidase using enzyme technology would result in color changes after the reaction of glucose oxidase and glucose in the blood, so that the user can interpret glucose concentration level based on comparison result of the color of the test strip with the color chart.

This simple blood glucose measurement method has been found beneficial to many patients with diabetes, such as monitoring disease at any time in terms of improved quality of life as well as expanded scope of medical care. However, the convenience of the test strip is still room for improvement since the test strip is still in need of special carry. Therefore, the development of a testing platform, which has the advantages of the test paper and has the convenience of matching with the daily necessities, is a current goal.

### SUMMARY OF THE INVENTION

The present invention is directed to providing a detection platform, which has advantages of test paper and is compatible to daily necessities.

According to one embodiment of the present invention, a lignocellulosic detection device includes a lignocellulosic substrate essentially made of a lignocellulosic tissue; at least one detection reagent coated onto the lignocellulosic tissue of the lignocellulosic substrate, whereby a liquid specimen is in contact with the lignocellulosic substrate so as to absorb to the lignocellulosic tissue of the lignocellulosic substrate by capillary action and react with the detection reagent to achieve specimen detection.

In one embodiment, the lignocellulosic substrate is a stirring rod, a toothpick or a wooden chopstick.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

FIG. 1 is a schematic diagram illustrating a lignocellulosic detection device of the present invention;

FIGS. 2a to 2c are the experimental datum showing a toothpick according to one embodiment of the present invention used for the measurement of glucose;

FIGS. 3a to 3c are the experimental datum showing a toothpick according to one embodiment of the present invention used for the measurement of cholesterol;

FIG. 4 is the experimental data showing a toothpick according to one embodiment of the present invention used for the measurement of nitrite;

FIG. 5 is a photo showing a stirring rod with surface treatment according to one embodiment of the present invention; and

FIG. 6 is the experimental data showing a stirring rod according to one embodiment of the present invention used for the measurement of nitrite.

### DESCRIPTION OF THE PREFERRED EMBODIMENT

The present invention relates to a lignocellulosic detection device provided with a lignocellulosic substrate and a detection reagent as the main component. Lignocellulosic substance, also called lignified xylem tissue or wood, refers to a collective plant tissue formed with vascular cambium inward development. In addition, the lignocellulosic is usually taken from the lignified xylem tissue formed in the trees and shrubs. The lignocellulosic substrate has a robust mechanical structure and relatively good resistance to acids or alkaline, and is provided with advantages in structural strength in comparison to filter papers.

The shape and/or size of the lignocellulosic substrate are not particularly limited in principle and may be mainly subjected to design requirement. Some common shapes of the lignocellulosic substrate may include without limitations to cylindrical, rectangular, plate-like shapes.

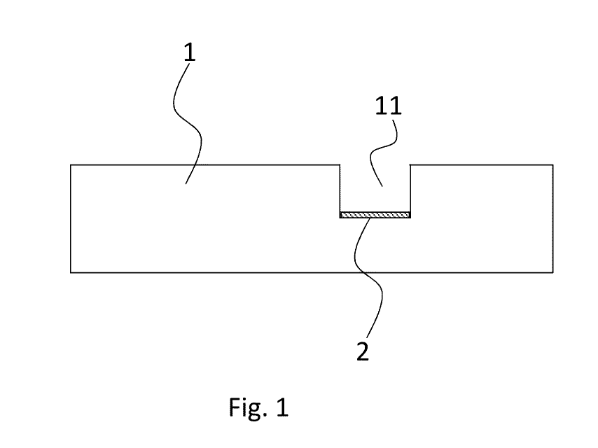

Referring to FIG. 1, which is schematically shows the lignocellulosic detection device of the present invention, wherein the lignocellulosic substrate 1 has at least one groove 11. Generally speaking, the shape and/or of the groove are not particularly limited in principle and may be mainly subjected to design requirement. It is noted that the shape of the groove 11 shown in FIG. 1 is only illustrative and not thus limited. Generally speaking, the shape of the groove 11 may include without limitations to cuboid, cube, cylinder, hemisphere, V-shape, other shapes, or combinations thereof. The position of the groove 11 relative to the lignocellulosic substrate 1 may also depend on the needs of different requirement for detection and be configured in a flexible way.

In one embodiment, the groove 11 may be set as the detection zone, and the detection reagent 2 therefore is provided in the groove 11 to carry out the detection. In FIG. 1, the detection reagent 2 is set at the bottom of the groove 11. It is understood, however, the detection reagent can also be set to single side, the bottom, bilateral sides of the groove or other combinations. In one embodiment, the liquid specimen can be dropped to the detection zone directly so as to react with the detection reagent and detect the liquid specimen.

It is noted that, in another embodiment, the internal transport channels (not shown) provided in the lignocellulosic substrate 1 may be used for transporting subject matter to be detected to the detection zone in the groove 11 via capillary action. The above-mentioned means provides some advantages such as avoiding measurement errors caused by physical damages of external transport channels caused by machining process, or achieving the goal of controlled flow rate of liquid specimen.

In addition, the lignocellulosic substrate may be subjected to surface treatment so as to increase the stability of the lignocellulosic fiber. Surface treatments may include without limitations to waterproof treatment, where waterproof reagents may include without limitations to PDMS (Polydimethylsiloxane).

It is also noted that multiple assays includes multiple qualitative and/or quantitative assays. To be specific, multiple assays may contain assays for various detecting subject matters or assays for a single detection subject matter of multiple concentrations, in order to achieve detection diversity.

In one embodiment, daily necessities can be used as the lignocellulosic substrate of the present invention. Examples may include without limitations to stirring rods, wooden chopsticks or toothpicks.

In one embodiment of the present invention, toothpicks may be adapted as the lignocellulosic substrate. The toothpicks are commonly used to clean food stuck in the teeth gap as its general purpose and usually made of lignocellulosic materials including bamboo. Bamboo plants are shrubs or trees having lignocellulosic pole. The bamboo materials have advantages in cost due to rapid growth of bamboo plants.

Therefore, toothpicks have advantages such as natural materials, low cost and easy machining process and so on. Toothpicks are easily available and commonly used in the postprandial oral hygiene maintenance. One purposes of the present invention is directed for users to use a toothpick to clean the teeth, together with the completion of disease detection. In such a way, users do not need to spend extra time for achieving disease detection.

The present invention is principally directed to using the lignocellulosic substrate having lignocellulosic tissue provided with capillary action and the detection reagent adsorbed on the fibrous tissue of the lignocellulosic substrate to function as a disease detection platform. When a liquid specimen or a specimen containing liquid is in contact with one end of the lignocellulosic substrate, the subject specimen is adsorbed to the lignocellulosic tissue of the lignocellulosic substrate by capillary action to react with the detection reagent so as to achieve the role of specimen detection.

The method of absorbing the detection reagent intothe lignocellulosic substrates may be achieved by soaking the lignocellulosic substrate with the detection reagent solution. The preparation is based on capillary action of the lignocellulosic substrate and vascular tissues of the plant fiber so as to make the detection reagent adsorbed onto the lignocellulosic substrate. Here, a drying step may be performed for obtaining a lignocellulosic substrate for detection after the lignocellulosic substrate is soaked in the detection reagent solution.

The lignocellulosic substrate for detection of the present invention may be detected by colorimetric reaction or fluorescence, accordingly the detection reagent comprises a colorimetric agent or a fluorescent reagent. Colorimetric agents or fluorescent reagents used as detection reagents may be chosen based on various subject matters targets to be detected and make adaptive changes.

In one embodiment of the present invention, the detection reagent may comprise an enzyme. As above-mentioned, qualitative measurements for detecting the presence of substances and quantitative measurements for determining the substance concentration may be performed by measuring changes in color or other properties caused by reaction between enzyme and substances in the solution. For example, color changes of the test strips using the glucose oxidase assay is achieved by the specific reaction catalyzed by glucose oxidase and glucose.

Examples of the specimen include without limitations to a subject's saliva, blood, urine or other body fluids. As mentioned above, the specimen may be absorbed to the fibrous tissue of the lignocellulosic substrate by capillary action to react with the detection reagent to achieve specimen detection. In addition, solid substances to be detected may be suspended and dissolved in a liquid solvent and then be detected by the above-described detection method using capillary action.

It is noted that the lignocellulosic substrates used in the present invention have been commonly used in eating or drinking. For example, lignocellulosic substrates such as toothpicks are used in postprandial oral hygiene maintenance; therefore, a preferred embodiment refers to the saliva of the test subjects. Advantages of saliva testing may include: non-invasiveness, convenient acquisition, rather low cost, low risk of infection, self-collection and self-test independent of medical staffs.

Continuing the above description, the lignocellulosic substrate for detection of the present invention may be used for testing biochemical properties, such as glucose, nitrite, pH, of saliva, blood, urine or other body fluids. The following examples are only used for illustrating the principle and applications of detection.

### EXAMPLE 1

### Nitrite Detection

In the case of nitrate ions present in the food, nitrosamines which have been identified as a carcinogen by the medical researchers are formed by action of digestive enzymes in the saliva. In addition, large amount of nitrite may cause direct poisoning after uptaking nitrite-abundant drinking water, vegetables, grain, fish, meat, salted products. At present, there are commercially available nitrite test strips using chemical colorimetric reaction; therefore, nitrite detection in food would be a suitable target for detection.

### EXAMPLE 2

### Glucose Detection

The measurement principle for blood sugar or urine glucose has been described above. Patients can use glucose detection device so as to monitor their conditions at any time.

### EXAMPLE 3

### PH Detection

Regarding the detection of pH, Litmus paper is commonly used to measure the pH of the solution. Litmus paper is made of paper immersed in the solution containing litmus reagent. The litmus reagent would indicate red in the acidic solution, and blue in the alkaline solution. The lignocellulosic substrate may be prepared using the aforementioned properties so as to detect the pH value.

The present invention is further illustrated by the following working examples, which should not be construed as further limiting.

### EXAMPLE 4

### Toothpick as Glucose Measuring Device

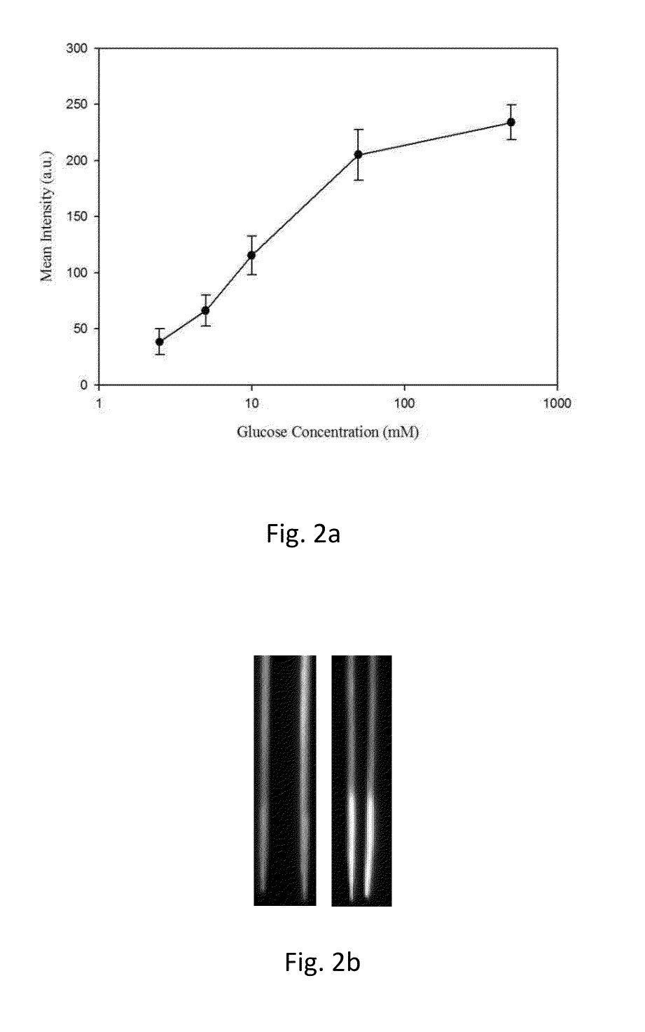

Preparation and assay methods using a toothpick for measuring glucose are listed as followings. (1) toothpick soaked in 330 μl of glucose detection solution containing: Glucose oxidase—75 units/mL, of HRP 15 units/mL, fluorescent dye 10-acetyl-3,7-dihydroxyphenoxazine 300 μM, all these drugs were from Sigma-Aldrich. (2) soaked the toothpick for five minutes. (3) removed the toothpick and dried for 2 hours. (4) the dried toothpicks were then ready for glucose detection. FIG. 2 shows the toothpicks adsorption results of different concentrations of glucose in the buffer system. Here, FIG. 2a is a calibration curve shows the corresponding relationship of the average fluorescence intensity and the concentration of glucose (N=20). FIG. 2b is a photo displaying fluorescence detection image of toothpick immersed in the buffer system containing 2.5 mM and 500 mM glucose, respectively. FIG. 2c is a diagram illustrating variations between different test groups of the same and different glucose concentration (each group n=5), wherein the average changes for test groups of glucose concentration 2. 5, 5, 10, 50 and 500 mM were respectively 0.633, 0.336, 0.118, 0.034 and 0.026.

### EXAMPLE 5

### Toothpick as Cholesterol Measurements Appliances

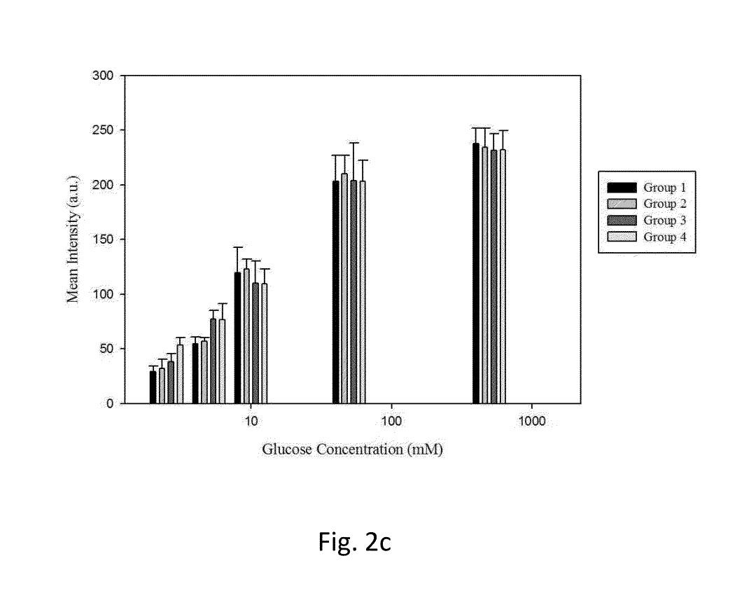

Preparation and assay methods using toothpicks for measuring cholesterol are listed as followings. (1) soaked toothpicks in the 330 μl cholesterol detection reagent containing cholesterol oxidase 2 units/mL, HRP units/mL, fluorescent dye 10-acetyl-3,7-dihydroxyphenoxazine 300 μM. All these drugs were obtained from Sigma-Aldrich. (2) soaked the toothpicks for five minutes. (3) removed the toothpicks and dried for 0.5 hour. (4) the dried toothpick were ready for cholesterol detection. FIG. 3 shows the toothpicks adsorption results of different concentrations of cholesterol in the buffer system. Here, FIG. 3a is a calibration curve shows the corresponding relationship of the average fluorescence intensity and the concentration of cholesterol (N=15). FIG. 3b is a photo displaying fluorescence detection image of toothpick immersed in the buffer system containing 1.5 mg/mL and 4 mg/mL cholesterol, respectively. FIG. 2c is a diagram illustrating variations between different test groups of the same and different glucose concentration (each group n=5), wherein the average changes for test groups of glucose concentration 1.5, 2, 2.5, 3 and 4 mg/mL were respectively 0.306, 0.177, 0.081, 0.088 and 0.107.

### EXAMPLE 6

### Toothpick as Nitrites Detection Device

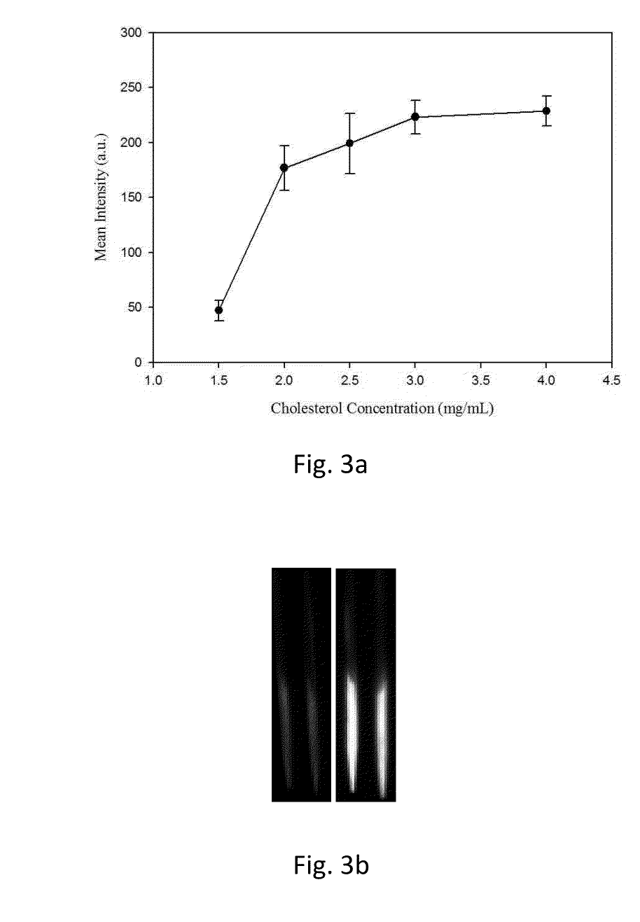

The preparation of nitrite detection device using toothpicks is listed as the following. Toothpicks are coated with a nitrite detection reagent containing 50 mmol/L sulfanilamide (≧99%, Sigma-Aldrich), 330 mmol/L citric acid (≧99.5%, Sigma-Aldrich), and 10 mmol/L N-(1-naphthyl) ethylenediamine) (≧98%, Sigma-Aldrich). FIG. 4 is a photo displaying detection image of toothpick immersed in the nitrite detection reagent and then exposed to nitrite solution of different concentration. The result is obtained from different nitrite concentration of 10, 5, 2.5, 1.25, 0.625 and 0.156 mM from left to right, respectively.

### EXAMPLE 7

### Surface Treatment for the Stirring Rod

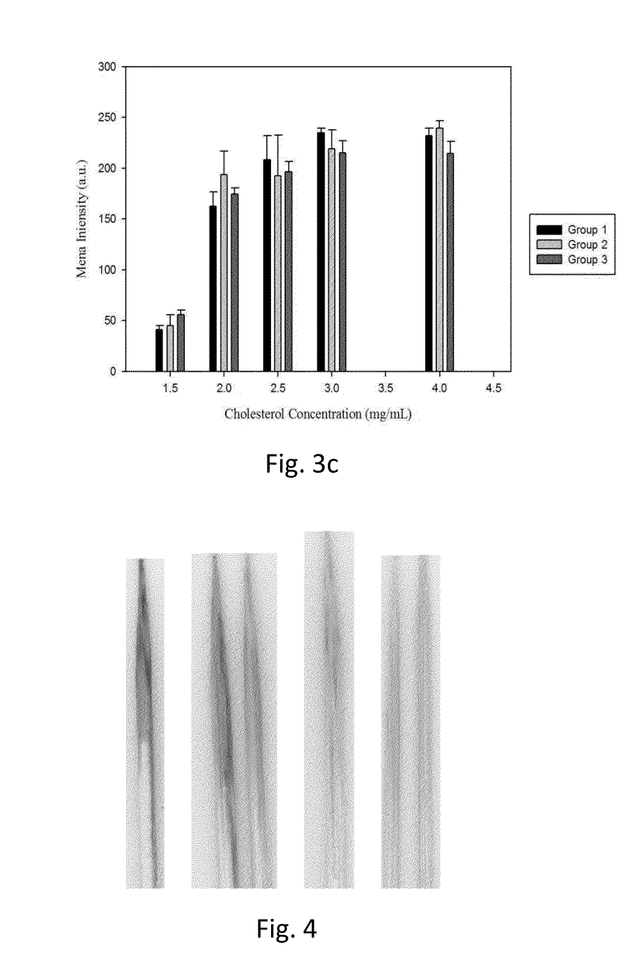

The surface treatment of the stirring rod for water proof is achieved by PDMS coating. One end of the stirring rod was then immersed in the red ink. Referring to FIG. 5, the result shows that the PDMS-coated stirring rods are water-proof in its surface, i.e. the red ink was transported internally to the detection zone in the groove. As for the control group, the surface of the control group was filled with red ink, namely passage of the red ink at its surface.

### EXAMPLE 8

### Stirring Rod as Glucose Measuring Device

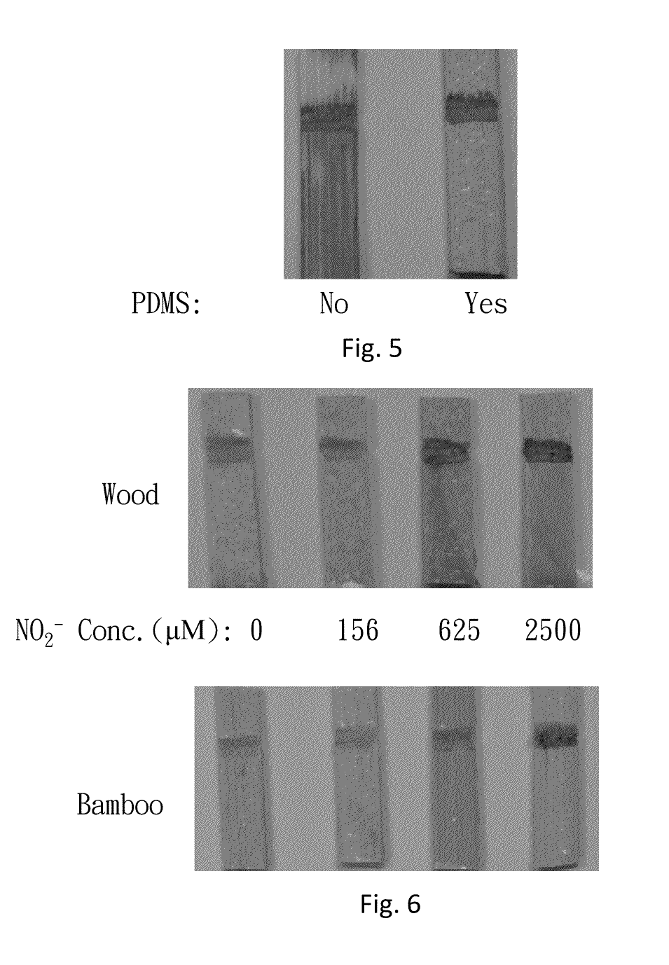

Wood and bamboo stirring rods were provided and subjected to surface treatment with 4 μl PDMS. 5 μl of nitrite detection reagent were dropped within the groove, where the nitrite detection reagent contains 50 mmol/L sulfanilamide, 330 mmol/L citric acid (≧99.5%, Sigma-Aldrich), 10 mmol/L N-(1-naphthyl) ethylenediamine (≧98%, Sigma-Aldrich). Refer to FIG. 6, which shows that wood and bamboo the detection reagent stirring rod coater Papua nitrate and nitrite solution with different concentrations of the reaction, the nitrite concentration, respectively, from left to right of 0, 156, 625, 2500 μM. FIG. 6 is a diagram illustrating wood and bamboo stirring rods coated with the nitrite detection reagent and then exposed to nitrite solution of different concentration. The result is obtained from different nitrite concentration of 0, 156, 625 and 2500 μM from left to right, respectively.

In summary, the advantages of the lignocellulosic detection device of the present invention include natural materials, low cost and easy machining process and could be used as a disease diagnosis platform or substrate. In addition, by using the detection device of the present invention, user could achieve disease diagnosis without spending extra time.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit and scope of the appended claims.

What is claimed is:

1. A lignocellulosic detection device, comprising:
a lignocellulosic substrate essentially made of a lignocellulosic tissue;
at least one detection reagent coated into the lignocellulosic tissue of the lignocellulosic substrate, whereby a liquid specimen is in contact with the lignocellulosic substrate so as to be absorbed to the lignocellulosic tissue of the lignocellulosic substrate by capillary action and react with the detection reagent to achieve specimen detection.

2. The lignocellulosic detection device as claimed in claim 1, wherein the lignocellulosic substrate is a stirring rod, a toothpick or a wooden chopstick.

3. The lignocellulosic detection device as claimed in claim 1, wherein the lignocellulosic substrate is made of bamboo.

4. The lignocellulosic detection device as claimed in claim 1, wherein the lignocellulosic substrate has a groove.

5. The lignocellulosic detection device as claimed in claim 4, wherein the detection reagent is configured in the groove.

6. The lignocellulosic detection device as claimed in claim 4, wherein the shape of the groove is cuboid, cube, cylinder, hemisphere, V-shape, or combinations thereof.

7. The lignocellulosic detection device as claimed in claim 1, wherein the detection reagent comprises a colorimetric agent or a fluorescent reagent.

8. The lignocellulosic detection device as claimed in claim 1, wherein the detection reagent comprises an enzyme.

9. The lignocellulosic detection device as claimed in claim 1, wherein the fluid specimen is saliva, blood or urine from a subject.

10. The lignocellulosic detection device as claimed in claim 1, being a glucose, nitrite, or pH detection device.

11. The lignocellulosic detection device as claimed in claim 1, wherein the surface of the lignocellulosic substrate is processed to be waterproof.

12. The lignocellulosic detection device as claimed in claim 1, wherein the liquid specimen is in contact with one end of the lignocellulosic substrate.

13. The lignocellulosic detection device as claimed in claim 1, wherein the lignocellulosic substrate is made of wood.
